(19) United States
(12) Patent Application Publication
(10) Pub. No.: US 2026/0232638 A1
(43) Pub. Date: Aug. 13, 2026

(54) COMPOUNDS FOR TREATING BRAIN CANCER

(71) Applicant: Purdue Pharmaceutical Products L.P., Stamford, CT (US)

(72) Inventors: Thomas Jorg Mehrling, Hunenberg (CH); Claudio Festuccia, L'Aquila (IT)

(73) Assignee: Purdue Pharmaceutical Products L.P., Stamford, CT (US)

(21) Appl. No.: 19/282,681

(22) Filed: Jul. 28, 2025

Related U.S. Application Data
(63) Continuation of application No. 17/874,621, filed on Jul. 27, 2022, now Pat. No. 12,370,177.
Application No. 17/874,621 is a continuation of application No. 15/314,172, filed on Mar. 26, 2015, now Pat. No. 11,419,853.

(30) Foreign Application Priority Data
May 28, 2014 (GB) 1409471.8

(51) Int. Cl. A61K 31/4184; A61K 31/407; A61K 31/427; A61K 31/502; A61K 31/5377; A61K 31/55; A61K 31/573; A61K 31/58; A61K 31/69; A61K 38/05; A61K 38/06; A61K 39/395; A61K 45/06

(52) U.S. Cl. CPC A61K 31/4184; A61K 31/407; A61K 31/427; A61K 31/502; A61K 31/5377; A61K 31/55; A61K 31/573; A61K 31/58; A61K 31/69; A61K 38/05; A61K 38/06; A61K 39/3955; A61K 45/06

(57) ABSTRACT

There is provided a compound of formula I or a pharmacologically acceptable salt thereof:

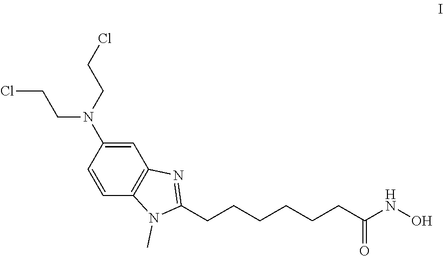 [SMILES: Cn1c(CCCCCCC(=O)NO)nc2cc(N(CCCl)CCCl)ccc21]

for use in the treatment of a brain cancer selected from a MGMT positive astrocytic brain tumor, a metastatic brain cancer and primary CNS lymphoma and a method of treating said brain cancers in a patient in need thereof comprising administering to the patient said compound of formula I or a pharmacologically acceptable salt thereof.

### CROSS-REFERENCE TO RELATED APPLICATIONS

This application is a continuation application of U.S. application Ser. No. 17/874,621, filed on Jul. 27, 2022, which is a continuation application of U.S. application Ser. No. 15/314,172, filed on Nov. 28, 2016, now U.S. Pat. No. 11,419,853, issued on Aug. 23, 2022, which is a U.S. national stage filing under 35 U.S.C. § 371 (c) of International Application No. PCT/EP2015/056667, filed on Mar. 26, 2015, which claims foreign priority to U.K. Patent Application No. 1409471.8, filed on May 28, 2014. The entire contents of each of the aforementioned applications are incorporated herein by reference.

### FIELD OF THE INVENTION

The present invention relates to novel treatments of brain cancers that have been particularly resistant to treatment in the past, namely astrocytic brain tumours, brain cancers that are metastasized cancers and primary CNS lymphomas.

### BACKGROUND TO THE INVENTION

Cancer is one of the most life threatening diseases. Cancer is a condition in which cells in a part of the body experience out-of-control growth. According to latest data from American Cancer Society, it is estimated there were 1.67 million new cases of cancer in USA in 2014. Cancer is the second leading cause of death in the United States (second only to heart disease) and it is estimated to have claimed more than 585,000 lives in 2014. In fact, it is estimated that 50% of all men and 33% of all women living in the United States will develop some type of cancer in their lifetime. Therefore cancer constitutes a major public health burden and represents a significant cost in the United States. These figures are reflected elsewhere across most countries globally, although the types of cancer and relative proportions of the population developing the cancers vary depending upon many different factors such including genetics and diet.

The World Health Organisation (WHO) classifies the primary brain tumours in four categories. WHO grade I and II are low-grade gliomas, whereas anaplastic astrocytomas and anaplastic oligodendrogliomas (WHO grade III), as well as glioblastomas (GBMs) (WHO grade IV), are collectively referred to as malignant gliomas. The prognosis of most primary and secondary brain tumours is abysmal due to lack of effective therapeutic agents. They are the leading cause of death from solid tumours in children and the third leading cause of death from cancer in adolescents and adults aged 15-34 years (Jemal et al, CA Cancer J Clin 59 2009 225-249).

Among the malignant gliomas, GBMs are the most common and fatal neoplasms, representing approximately 50% of all gliomas. GBM has a dismal prognosis, highlighting the need for novel treatment strategies. Surgery followed by a combined therapy of the alkylating agent temozolomide (TMZ) and radiotherapy is the standard treatment for patients suffering from GBM. The principal mechanism of action of TMZ is initiated by abnormal methylations of DNA bases, particularly 06-methylguanine in DNA (Verbeek et al, Br Med Bul, 85, 2008, 17-33). However, many patients are resistant or show only weak reaction to TMZ. This has been shown to be conferred by 06-methylguanine-DNA methyltransferase (MGMT) mediated mismatch repair (MMR) (see Weller et al, Nat Rev Neurol, 6, 2010, 39-51). Patients having this repair system have ‘MGMT positive GBMs.’ The activation of mTOR/DNAPKC pathways is also believed to play a part. No chemotherapeutic agents have been developed to date which are active against MGMT positive GBMs. The activity of MGMT is also important in other astrocytic brain tumours, namely diffuse astrocytomas (WHO grade II) and anapalastic astrocytomas (WHO grade III). Progression of these to GBMs is primarily mediated through methylation by MGMT. It can therefore be seen that a therapeutic agent which is active against MGMT positive astrocytomas will be desirable in preventing advance of these diffuse and anaplastic astrocytomas to GBMs.

It is therefore essential that a novel therapeutic agent with an excellent anti-neoplastic activity against not only against MGMT negative GBMs but also against MGMT positive GBMs (as well as other astrocytic brain tumours), which shows excellent CNS penetration and has a tolerable toxicity profile is urgently developed.

A metastatic brain tumour starts as a cancer elsewhere in the body and spreads to the brain. Breast, lung, melanoma, colon and kidney cancers commonly metastatize. Frequently, the metastatic brain tumour is discovered before the primary tumour. Metastatic brain tumours are the most common of all brain tumours in adults. It is estimated that there may be up to 170,000 new cases each year. Although a little better than for GBMs, the prognosis for metastatic brain cancers is generally poor. Once again, a combination of surgery, therapy and chemotherapy is adopted, with the exact combination from within these options being dependent upon the nature of the metastatic cancer and the stage of development (as well as the health of the patient). Surgery (where possible) and radiotherapy is the standard treatment applied. Chemotherapy is sometimes employed. Unfortunately, none to date have been very successful. Part of this is due to the need for the chemotherapeutic agent to show excellent CNS penetration (as well, of course, as excellent anti-neoplastic activity and a tolerable toxicity profile). Many existing chemotherapeutic agents show a poor penetration across the blood-brain barrier. There is an urgent need for a novel therapeutic agent that addresses these problems.

Primary central nervous system (CNS) lymphoma originates in the lymphocytes but should be considered a brain tumour because its location is solely in the brain and the therapeutic challenges resemble those of other brain tumours. In particular, drug delivery is impaired by the blood-brain barrier and cerebral toxicity limits the use of current treatments. Most primary CNS lymphomas are diffuse large B-cell lymphomas (about 90%). Although it is relatively rare, its incidence and prevalence are increasing. Currently, the median survival rate with existing treatment regimes is 44 months. No particularly effective treatment regimen has yet been established for this condition. The current preferred chemotherapeutic agent is methotrexate. However, its penetration across the blood-brain barrier is not satisfactory and it has to be administered in very high doses. Combination therapy with radiotherapy can improve outcomes, but side effects can be very severe. There is therefore a need for an improved chemotherapeutic agent which has a greater ability to penetrate the blood-brain barrier and also shows excellent anti-neoplastic activity against primary CNS lymphomas.

In WO-A-2010/085377, the compound of formula I below is disclosed. It is a first-in-class dual-functional alkylating-HDACi fusion molecule which potently inhibits the HDAC pathway.

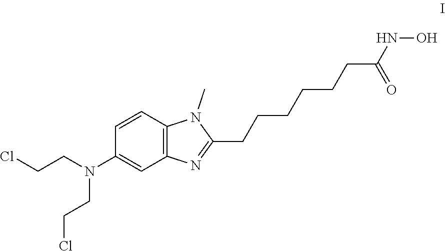 [SMILES: Cn1c(CCCCCCC(=O)NO)nc2cc(N(CCCl)CCCl)ccc21]

Biological assays showed that the compound of formula I potently inhibits class 1 and class 2 HDAC enzymes (e.g. HDAC1 IC₅₀ of 9 nM) and it has been shown to have excellent in vitro activity against multiple myeloma cell lines. Moreover, it suppresses DNA repair via significant downregulation of FANCD2, BRCA1, BRCA2, and TS (Thymidylate synthetase), possibly related to HDAC6 and HDAC8 inhibition. Cytotoxicity assay in NCI-60 cell lines has shown that it has a very potent anticancer activity with a median IC₅₀ value of 2.2 μM compared to 72 μM for Bendamustine. WO-A-2013/113838 includes data that demonstrates the activity of the compound of formula I (referred to as NL-101 in the description) against a number of cell lines, including some glioblastoma cell lines. However, each of the cell lines in question is a MGMT negative GBM tumour cell line.

### SUMMARY OF THE INVENTION

In a first aspect of the present invention there is provided a compound of formula I or a pharmacologically acceptable salt thereof:

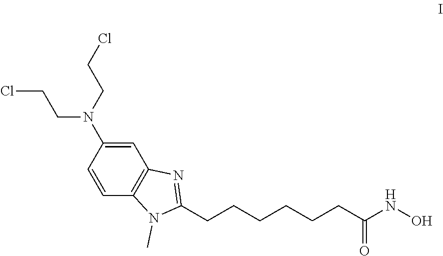 [SMILES: Cn1c(CCCCCCC(=O)NO)nc2cc(N(CCCl)CCCl)ccc21]

for use in the treatment of a brain cancer selected from a MGMT positive astrocytic brain tumour, a metastatic brain cancer and primary CNS lymphoma.

In pre-clinical in vitro and in vivo studies it has been shown that the compound of formula I is active against not only MGMT negative GBM tumours but also MGMT positive GBM tumours. From this, it can also be expected that it would be active against other MGMT positive astrocytic tumours. It has also been found that the compound of formula I is able to penetrate the blood-brain barrier very well, making it ideal for therapeutic use against not only MGMT positive astrocytic tumours, but also other brain tumours. In particular, it has been further found to have very good activity against metastatic brain cancer and also primary CNS lymphoma.

In a second aspect of the present invention there is provided use of a compound of formula I or a pharmacologically acceptable salt thereof in the manufacture of a medicament for the treatment of a brain cancer selected from a MGMT positive astrocytic brain tumour, a metastatic brain cancer and primary CNS lymphoma.

In a third aspect of the present invention there is provided a method of treating a brain cancer selected from a MGMT positive astrocytic brain tumour, a metastatic brain cancer and primary CNS lymphoma in a patient in need thereof comprising administering to said patient a compound of formula I or a pharmacologically acceptable salt thereof.

### DESCRIPTION OF THE DRAWINGS

FIG. 1 is a plot of EDO-S101 concentration (μM) versus time in the cerebrospinal fluid and the blood versus time;

FIG. 2 is a plot of the IC₅₀ for the twelve tested GBM cell lines after temozolomide was administered;

FIG. 3 is a plot of the IC₅₀ for the twelve tested GBM cell lines after temozolomide and vorinostat was administered;

FIG. 4 is a plot of the IC₅₀ for the twelve tested GBM cell lines after bendamustine was administered;

FIG. 5 is a plot of the IC₅₀ for the twelve tested GBM cell lines after bendamustine and vorinostat was administered;

FIG. 6 is a plot of percentage of cell survival against concentration of EDO-S101 (μM) for each of the twelve tested cell lines;

FIG. 7a is a plot of luminescence against time as a measure of growth of GBM12 cells post-injection;

FIG. 7b is a plot of percent survival versus time showing the prolongation of survival for EDO-S101 against bendamustine and control;

FIG. 8 is a plot of time to progression (TTP) probability (%) against time for mice having implanted U251 tumours treated with EDO-S101;

FIG. 9 is a plot of time to progression (TTP) probability (%) against time for mice having implanted U87 tumours treated with EDO-S101;

FIG. 10 is a plot of surviving fraction against the dose of radiotherapy (Gy) for U251, U87 and T98G cells treated with radiotherapy alone, radiotherapy and 2.5 μM EDO-S101 (shown as NL-101 in the figure) and 5 μM EDO-S101 EDO-S101;

FIG. 11 is a plot of time to progression (TTP) probability (%) against time for mice having implanted U251 tumours treated with control, radiotherapy and EDO-S101;

FIG. 12 is a plot of time to progression (TTP) probability (%) against time for mice having implanted U251 tumours treated with control, radiotherapy and temozolomide, EDO-S101, and Radiotherapy and EDO-S101;

FIG. 13 is a plot of time to progression (TTP) probability (%) against time for mice having implanted U87 tumours treated with control, radiotherapy and EDO-S101;

FIG. 14 is a plot of time to progression (TTP) probability (%) against time for mice having implanted U87 tumours treated with control, radiotherapy and temozolomide, EDO-S101, and radiotherapy and EDO-S101;

FIGS. 15 and 16 are bioluminescence images of orthotopic luciferase-transfected U251 GBM mice, after treatment with control vehicle, EDO-S101, temozolomide, and radiotherapy and temozolomide;

FIG. 17 is a plot of survival probability (%) against time for orthotopic luciferase-transfected U251 GBM mice, after treatment with control vehicle, radiotherapy, EDO-S101, temozolomide, and radiotherapy and temozolomide;

FIG. 18 is a plot of percent survival against time for mice having implanted OCI-LY10 CNS lymphomas treated with control, bendamustine and EDO-S101; and

FIG. 19 is a plot of percent survival against time for mice having triple negative metastatic breast cancer of the brain after transfection with MB-468 breast cancer cells treated with control, bendamustine and EDO-S101.

### DETAILED DESCRIPTION OF THE INVENTION

In the present invention, a number of general terms and phrases are used, which should be interpreted as follows.

An astrocytic brain tumour is a tumour derived from star-shaped glial cells (astrocytes) in the brain. They are divided into low grade (I and II) and high grade (III and IV). Grade II astrocytic tumours are known as diffuse astrocytomas. Although these grow relatively slowly they can develop into malignant primary tumours. Grade III astrocytic tumours are known as anaplastic astrocytomas. These are malignant tumours; they grow more rapidly and tend to invade nearby healthy tissue. Grade IV astrocytic tumours are known as glioblastoma multiforme (GBM). These are highly malignant, growing rapidly, spreading readily to nearby tissue and are very difficult to treat with conventional treatments.

The current standard chemotherapeutic treatment is with temozolomide (TMZ). However, many patients are resistant or show only weak reaction to reaction. This has been shown to be conferred by 06-methylguanine-DNA methyltransferase (MGMT) mediated mismatch repair (MMR) (see Weller et al, Nat Rev Neurol, 6, 2010, 39-51). Patients having this repair system have ‘MGMT positive GBMs.’ GBMs are thus divided up as MGMT negative GBMs and MGMT positive GBMs depending upon whether they express the MGMT gene. The compounds of formula I of the present invention or a pharmacologically acceptable salt thereof have been shown to be active against not only MGMT negative GBMs but also MGMT positive GBMs.

The activity of MGMT is also important in other astrocytic brain tumours, namely diffuse astrocytomas (WHO grade II) and anapalastic astrocytomas (WHO grade III). Progression of these to GBMs is primarily mediated through methylation by MGMT. It can therefore be seen that as the compound of formula I and pharmacologically salts thereof are active against MGMT positive astrocytomas, it will also be capable of preventing advance of these diffuse and anaplastic astrocytomas to GBMs.

A metastatic brain tumour is a brain tumour that starts as a cancer elsewhere in the body and spreads to the brain. Breast, lung, melanoma, systemic lymphoma, sarcoma, colon, gastro-intestinal and kidney cancers commonly metastasize.

A primary CNS lymphoma in the context of the present invention is a lymphoma that originates in the lymphocytes in the brain, malignant cells formed from said lymphocytes. It is hence considered a brain tumour because its location and therapeutic challenges resemble those of other brain tumours.

“Pharmaceutically acceptable salts” means salts of compounds of the present invention which are pharmaceutically acceptable, as defined above, and which possess the desired pharmacological activity. Such salts include acid addition salts formed with inorganic acids, or with organic acids. Pharmaceutically acceptable salts also include base addition salts which may be formed when acidic protons present are capable of reacting with inorganic or organic bases. Generally, such salts are, for example, prepared by reacting the free acid or base forms of these compounds with a stoichiometric amount of the appropriate base or acid in water or in an organic solvent or in a mixture of the two. Generally, non-aqueous media like ether, ethyl acetate, ethanol, isopropanol or acetonitrile are preferred. Examples of the acid addition salts include mineral acid addition salts such as, for example, hydrochloride, hydrobromide, hydroiodide, sulfate, bisulfate, sulfamate, nitrate, phosphate, and organic acid addition salts such as, for example, acetate, trifluoroacetate, maleate, fumarate, citrate, oxalate, succinate, tartrate, salicylate, tosylate, lactate, naphthalenesulphonate, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

In the present invention, the pharmacologically acceptable salt of the compound of formula I may preferably be the hydrochloride, hydrobromide, hydroiodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate or acetate, and more preferably the acetate.

In the present invention, when the compound of formula I or a pharmacologically acceptable salt thereof is for use in the treatment of a MGMT positive astrocytic brain tumour, this is preferably selected from a MGMT positive glioblastoma multiforme, a diffuse (WHO grade II) astrocytoma and an anaplastic (WHO grade III) astrocytoma, and most preferably a MGMT positive glioblastoma multiforme.

In the present invention, when the compound of formula I or a pharmacologically acceptable salt thereof is for use in the treatment of a metastatic brain cancer, this is preferably selected from metastasized breast cancer, metastasized systemic lymphoma, metastasized lung cancer, metastasized melanoma, metastasized sarcoma and metastasized gastro-intestinal cancer, and most preferably metastasized breast cancer.

The therapeutically effective amount of the compound of formula I or a pharmacologically acceptable salt and the medicament comprising it administered to the patient according to the first, second and third aspects of the present invention is an amount which confers a therapeutic effect in accordance with the present invention on the treated subject, at a reasonable benefit/risk ratio applicable to any medical treatment. The therapeutic effect may be objective (i.e. measurable by some test or marker) or subjective (i.e. subject gives an indication of or feels an effect). An effective amount of the compound of formula I or a pharmacologically acceptable salt thereof according to the present invention is believed to be one wherein the compound of formula I or a pharmacologically acceptable salt thereof is included at a dosage range of from 0.1 to 70 mg/kg body weight patient (e.g. 0.5 to 50 mg/kg body weight such as 1, 5, 10, 20, 30, 40 or 50 mg/kg body weight).

The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

Suitable examples of the administration form of the compound of formula I or a pharmacologically acceptable salt thereof and medicament comprising the same according to the first, second and third aspects of the present invention include without limitation oral, topical, parenteral, sublingual, rectal, vaginal, ocular, and intranasal. Parenteral administration includes subcutaneous injections, intravenous, intramuscular, intrasternal injection or infusion techniques. Preferably, the compound of formula (I) or a pharmacologically acceptable salt thereof and medicament comprising the same are administered parenterally, and most preferably intravenously.

Preferably, the compound of formula I or a pharmacologically acceptable salt thereof is administered intravenously to the patient in need thereof at a dosage level to the patient in need thereof of from 0.1 mg/kg to 70 mg/kg body weight patient, and most preferably intravenously to the patient in need thereof at a dosage level of from 0.5 mg/kg to 50 mg/kg body weight patient.

It has been found that in the first, second and third aspects of the present invention, the compound of formula I or a pharmacologically acceptable salt thereof or medicament comprising the same may preferably be administered to a patient in need thereof on days 1, 8 and 15 of a treatment cycle, on days 1 and 8 of a treatment cycle or day 1 only of a treatment cycle.

In another preferred embodiment of the first, second and third aspects of the present invention it has surprisingly been found that the compound of formula I and pharmacologically acceptable salts thereof are considerably more effective when administered in combination with radiotherapy, and indeed appear to be synergistic with radiotherapy both in in vitro and in vivo studies. As a consequence, in the first, second and third aspects of the present invention the compound of formula I or a pharmacologically acceptable salt thereof or the medicament comprising the same may be used in treatment of a patient in need thereof wherein the patient in need thereof is also given radiotherapy either prior to or after the treatment of the brain cancer with the compound of formula I or a pharmacologically acceptable salt thereof or the medicament comprising the same. Preferably, the patient is given radiotherapy treatment prior to the treatment with the compound of formula I or a pharmacologically acceptable salt thereof or the medicament comprising the same. The radiotherapy may be given at a dose of 1 to 5 Gy over 5 consecutive days and preferably 2 Gy over 5 consecutive days.

In a further preferred embodiment of the first, second and third aspects of the present invention, the treatment further comprises the administration to a patient in need thereof of a vascular endothelial growth factor (VEGF) inhibitor, and the compound of formula I or a pharmacologically acceptable salt thereof and the vascular endothelial growth factor (VEGF) inhibitor may be administered concurrently, sequentially or separately, and preferably concurrently. Preferably, the vascular endothelial growth factor (VEGF) inhibitor is bevacizumab.

In a further preferred embodiment of the first, second and third embodiments of the present invention, the treatment further comprises the administration to a patient in need thereof of a poly ADP ribose polymerase (PARP) inhibitor, and the compound of formula I or a pharmacologically acceptable salt thereof and the poly ADP ribose polymerase (PARP) inhibitor may be administered concurrently, sequentially or separately, and preferably concurrently. Preferably, the poly ADP ribose polymerase (PARP) inhibitor is selected from rucaparib, olaparib and veliparib.

In a further preferred embodiment of the first, second and third embodiments of the present invention, the treatment further comprises the administration to a patient in need thereof of a PD-1/PDL-1 (immune checkpoint) inhibitor, and the compound of formula I or a pharmacologically acceptable salt thereof and the PD-1/PDL-1 (immune checkpoint) inhibitor may be administered concurrently, sequentially or separately, and preferably concurrently. Preferably, the PD-1/PDL-1 (immune checkpoint) inhibitor is ipilimumab.

When intended for oral administration, the compound of formula I or a pharmacologically acceptable salt thereof or medicament comprising the same of the first, second and third aspects of the present invention may be in solid or liquid form, where semi-solid, semi-liquid, suspension and gel forms are included within the forms considered herein as either solid or liquid.

The compound of formula I or a pharmacologically acceptable salt thereof or medicament comprising the same of the first, second and third aspects of the present invention can be prepared for administration using methodology well known in the pharmaceutical art. Examples of suitable pharmaceutical formulations and carriers are described in “Remington's Pharmaceutical Sciences” by E. W. Martin.

As a solid composition for oral administration, the compound of formula I or a pharmacologically acceptable salt thereof or medicament comprising the same of the first, second and third aspects of the present invention can be formulated into a powder, granule, compressed tablet, pill, capsule, chewing gum, wafer or the like form. Such a solid composition typically contains one or more inert diluents, either as a single tablet comprising all active agents or as a number of separate solid compositions, each comprising a single active agent of the combination of the present invention (in the case of the kit). In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent.

When the compound of formula I or a pharmacologically acceptable salt thereof or medicament comprising the same of the first, second and third aspects of the present invention is in the form of a capsule (e. g. a gelatin capsule), it can contain, in addition to materials of the above type, a liquid carrier such as polyethylene glycol, cyclodextrin or a fatty oil.

The compound of formula I or a pharmacologically acceptable salt thereof or medicament comprising the same of the first, second and third aspects of the present invention can be in the form of a liquid, e. g. an elixir, syrup, solution, emulsion or suspension. The liquid can be useful for oral administration or for delivery by injection. When intended for oral administration, a compound of formula I or a pharmacologically acceptable salt thereof or medicament comprising the same of the first, second and third aspects of the present invention can comprise one or more of a sweetening agent, preservatives, dye/colorant and flavor enhancer. In a compound of formula I or a pharmacologically acceptable salt thereof or medicament comprising the same of the first, second and third aspects of the present invention for administration by injection, one or more of a surfactant, preservative, wetting agent, dispersing agent, suspending agent, buffer, stabilizer and isotonic agent can also be included.

The preferred route of administration is parenteral administration including, but not limited to, intradermal, intramuscular, intraperitoneal, intravenous, subcutaneous, intranasal, epidural, intranasal, intracerebral, intraventricular, intrathecal, intravaginal or transdermal. The preferred mode of administration is left to the discretion of the practitioner, and will depend in part upon the site of the medical condition (such as the site of cancer). In a more preferred embodiment, the compound of formula I or a pharmacologically acceptable salt thereof or medicament comprising the same of the first, second and third aspects of the present invention are administered intravenously.

The liquid compound of formula I or a pharmacologically acceptable salt thereof or medicament comprising the same of the first, second and third aspects of the present invention, whether they are solutions, suspensions or other like form, can also include one or more of the following: sterile diluents such as water for injection, saline solution, preferably physiological saline, Ringer's solution, isotonic sodium chloride, fixed oils such as synthetic mono or digylcerides, polyethylene glycols, glycerin, or other solvents; antibacterial agents such as benzyl alcohol or methyl paraben; and agents for the adjustment of tonicity such as sodium chloride or dextrose. A parenteral combination or composition can be enclosed in an ampoule, a disposable syringe or a multiple-dose vial made of glass, plastic or other material. Physiological saline is a preferred adjuvant.

The compound of formula I or a pharmacologically acceptable salt thereof or medicament comprising the same of the first, second and third aspects of the present invention of the present invention can be administered by any convenient route, for example by infusion or bolus injection, by absorption through epithelial or mucocutaneous linings, and preferably by bolus injection.

### EXAMPLES

In the following examples, the compound having the following formula I is referred to as EDO-S101.

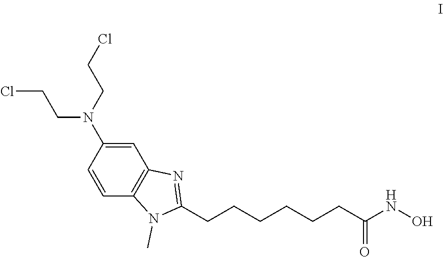 [SMILES: Cn1c(CCCCCCC(=O)NO)nc2cc(N(CCCl)CCCl)ccc21]

EDO-S101 was prepared as described in Example 6 of WO-A-2010/085377. EDO-S101 was dissolved in DMSO (100X mother solution) and stored at 4° C. before to be suspended in medium on the day of use.

### Example 1 CNS Pharmacokinetic Analysis of EDO-S101 in Sprague-Dawley Rats

CNS pharmacokinetics was determined in rats after a tail vein injection of EDO-S101 at 40 mg/kg. Microdialysate samples were collected from the blood and a ventricle of the brain via microdialysis probes at 18 time intervals. The drug concentrations in these samples were determined by capillary electrophoresis with UV detection (CE-UV) followed by calculations for various pharmacokinetic parameters.

Six rats were anesthetized with gasiform isoflurane (1% isoflurane in a mixture of 20% oxygen and 80% nitrogen gas) and immobilized in a stereotaxic frame (KOPF Instruments, Tujunga, CA). Anaesthesia was maintained during the entire procedure. Each guide cannula (CMA Microdialysis Inc., Acton, MA) was stereotactically implanted into the lateral ventricle (AP-0.9, L 1.6, V 3.4, relative to bregma and skull), then secured to the skull by screws and dental cement. Following surgery, each rat was housed individually with food and water ad libitum for 3 days for recovery from cannulation surgery. Microdialysis experiments were carried out on conscious, freely moving rat. On the day of the experiment, the stylet in the guide cannula was replaced with the microdialysis probe (CMA/11 with 4 mm membrane, CMA Microdialysis Inc., Acton, MA) and a vascular microdialysis probe (CMA/20 with 4 mm membrane, CMA Microdialysis Inc, Acton, MA)) was implanted into the jugular vein. The probes had inlet tubes connected to syringes to deliver artificial cerebrospinal fluid (146 mM NaCl, 1.2 mM CaCl₂), 3 mM KCl, 1 mM MgCl2, 1.9 mM Na₂HPO₄, 0.1 mM NaH₂PO₄, pH 7.4) into the ventricle and Dulbecco's phosphate-buffered saline (D-PBS) into the blood at 0.5 μl/min flow rate. The outlet tubes were connected to a microfraction collector for collecting the microdailysates at 4° C. The rats were allowed to recover for at least 24 hours prior to dosing. Eighteen samples were collected over 3 hours after EDO-S101 injection (intravenously). All samples were applied to the capillary electrophoresis with UV detection (CE-UV) for the determination of concentration of EDO-S101 in the cerebrospinal fluid (CSF) and blood. The rats were sacrificed using CO₂ inhalation after the experiment. The position of the probe was verified by visual inspection at the end of each experiment.

EDO-S101 in the microdialysate were measured by CE-UV (Agilent 3D CE). Briefly, the capillaries were preconditioned with 1 M sodium hydroxide for 2 min, water for 2 min and running buffer [100 mmol/l solution of ammonium acetate (adjust to pH 3.1 with acetic acid)-acetonitrile (50:50, v/v)] for 3 min. The samples were injected at a pressure of 0.7 psi for 5 s and the injection volume was approximately 5 nl. After injection, EDO-S101 was separated in a fused silica capillary of 50 μm I.D. and 50/65 cm length (effective length/total length) under 15 kv and 25° C. The absorbance from EDO-S101 was detected with UV at 300 nM. Emission was collected on a photomultiplier tube (PMT).

To perform a statistical analysis on the data, a two-way repeated measures ANOVA followed by Tukey's test was used. P<0.05 was considered significant. CNS penetration is determined as the ratio of CSF and blood area under the curve (AUC).

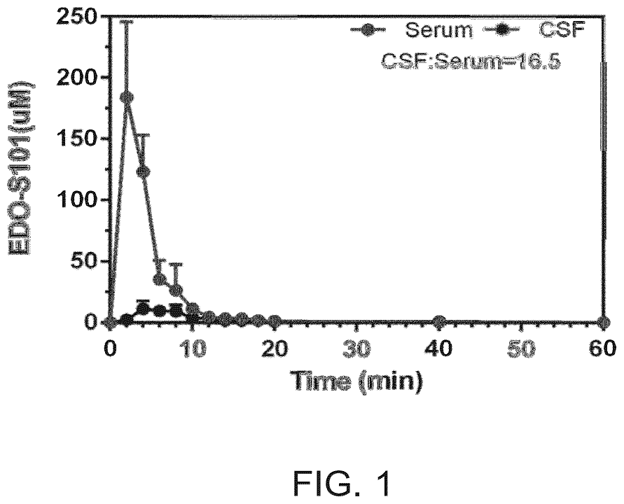

On analysing the results, it was found that EDO-S101 crosses the blood brain barrier well with a CNS penetration of 16.5% (see FIG. 1). It can achieve a high CNS concentration with a Cmax of 11.2 μM. As such, EDO-S101 is ideal for therapeutic use in brain tumours. It was also shown that it has a very short half-life of about 6 minutes in the blood and about 9 minutes in the brain. As the drug concentrations were determined based of the absorbance of EDO-S101 at UV wavelength of 300 nM, all the measurements are on the unmetabolized EDO-S101. The results are summarised in Table 1 as follows.

TABLE 1

| PK parameters | Blood | Brain |
|---|---|---|
| Cₘₐₓ (μM) | 184.0 ± 61.8 | 11.2 ± 6.5 |
| Tₘₐₓ (min) | 2.33 ± 0.82 | 5.67 ± 1.97 |
| T_{1/2} (min) | 5.6 ± 1.07 | 8.8 ± 1.43 |
| AUC (0-12) (μM · hr) | 824.3 ± 110.8 | 136.2 ± 74.7 |

| | |
|---|---|
| AUC ratio (Brain:Blood) | 16.5% ± 0.09 |

### Example 2 In Vitro Activity Tests for EDO-S101 and Known Compounds Against Various MGMT Positive and Negative Cell Lines

In vitro experiments were devised in which a series of GBM cell lines representative for MGMT negative and MGMT positive tumour cells were used.

Compounds: 1-100 μM EDO-S101, 1-50 UM temozolomide (TMZ), 1-50 μM temozolomide+500 nM vorinostat, 1-40 μM bendamustine, 1-40 μM bendamustine and 500 nM vorinostat.

Cell lines: A172, LN229, SNB19, SW1783, U251, U373 and U87: MGMT negative cell lines; LN18, Mz54, T98G, U138, U118: MGMT positive cell lines

Twelve glioblastoma cell lines representing grade III and IV gliomas and with different expression of MGMT, drug and radiotherapy sensitivity and five patient derived glioblastoma stem, cells were used (see above). Four patient-derived glioblastoma stem cells, kindly provided from J. Gregory Cairncross, and Samuel Weiss at the Hotchkiss Brain Institute, Faculty of Medicine, University of Calgary, Calgary, Alberta, Canada and one luciferase transfected, PTC #8, from Prof Angelo Vescovi, University Ia Bicocca, Milan were cultured in defined culture serum free medium (SFM) and in non-adherent spheres culture. Cells were re-suspended in DMEM/F12 media without serum supplemented with 20 ng/ml epidermal growth factor (Sigma-Aldrich), 20 ng/ml basic fibroblast growth factor (Sigma-Aldrich), B-27 supplement 1X (Gibco, Life Technologies), and antibiotics. Treatment with EDO-S101 was added straight after plating 3×10³ cells in 96-well plates with the stem cells media. Spheres were counted 5 days after treatment under an inverted microscope at ×4 magnification. A sphere was counted if it had at least 15 cells.

Cells were seeded at a density of 2×10⁴ cells/ml in 24 well plates. Cells were left to attach and grow in 5% FCS DMEM for 24 h. After this time, cells were maintained in the appropriate culture conditions. Morphological controls were performed every day with an inverted phase-contrast photomicroscope (Nikon Diaphot, Tokyo, Japan), before cell trypsinization and counting. Cells trypsinized and resuspended in 1.0 ml of saline were counted using the NucleoCounter™ NC-100 (automated cell counter systems, Chemotec, Cydevang, DK) in order to evaluate cell viability. All experiments were conducted in triplicate. IC₅₀ values were calculated by the GraFit method (Erithacus Software Limited, Staines, UK). Cell viability was measured with the 3-(4,5 dimethylthiazol-2-yl)-2,5-diphenyl tetrazolium bromide (MTT; Sigma-Aldrich) assay.

IC₅₀ and IC₂₀ values for all twelve cell lines against bendamustine and vorinostat were also determined as described above. Next, combination assays with fixed dose of vorinostat (IC₂₀ value) and varying the dose of bendamustine were performed. New IC₅₀ values were calculated for bendamustine when combined with vorinostat.

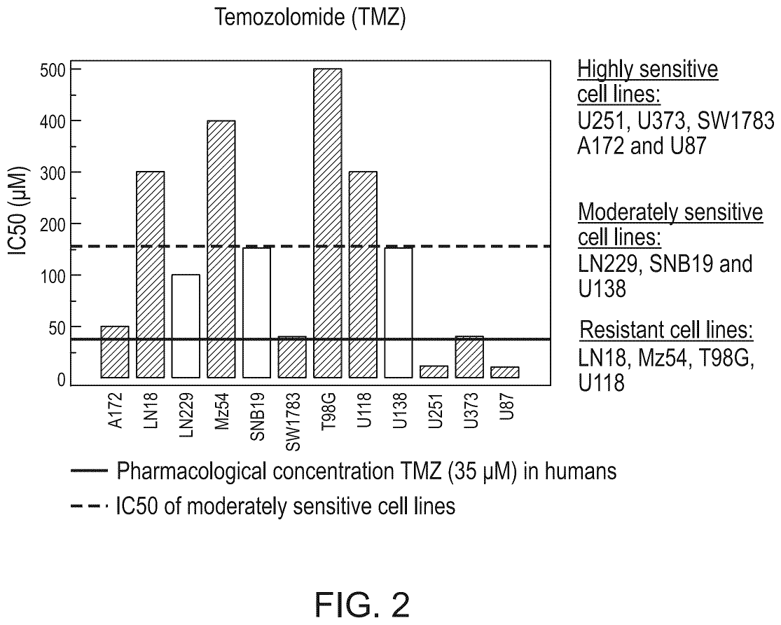

As can be seen from FIG. 2, the U251, U373, SW1783, A172 and U87 GBM cell lines are highly sensitive to TMZ, while LN229, SNB19 and U138 are moderately sensitive. The MGMT positive GBM cell lines LN18, Mz54, T98G and U118 were, however, resistant to TMZ.

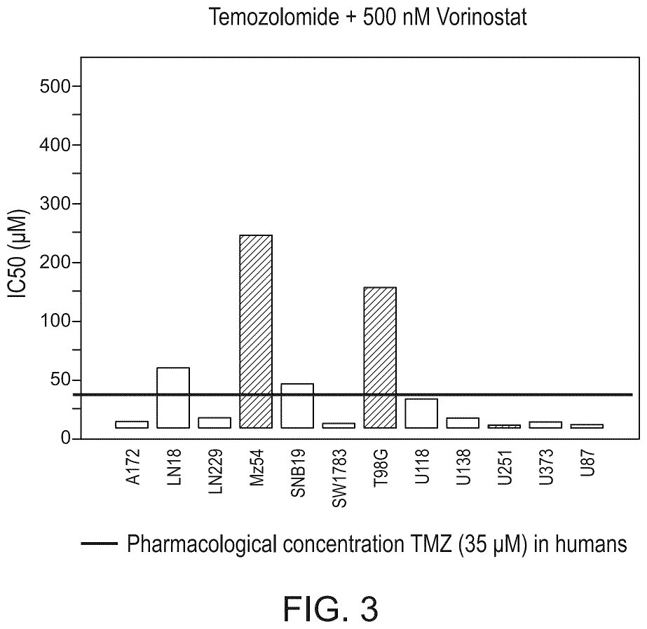

In a separate experiment, TMZ was used in combination with 500 nM vorinostat. It is known that vorinostat is synergistic with TMZ in GBM cell lines. As can be seen from FIG. 3, while the MGMT positive GBM cell lines LN18 and U118 were sensitive to this combination, T98G and Mz54 were still very resistant. The IC₅₀ of T98G was reduced but it is not the range of achievable doses in humans.

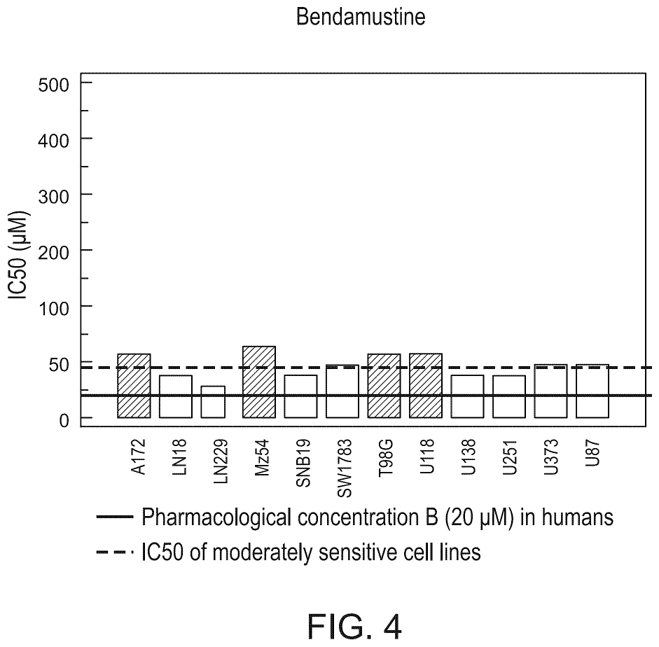

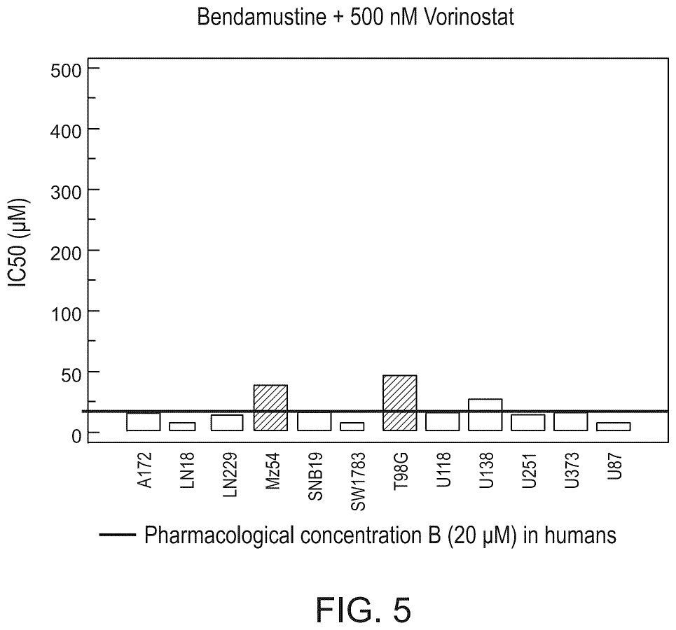

FIG. 4 shows that none of the GBM cell lines were highly sensitive to bendamustine, while LN18, LN229, SNB19, U138, U251, U373, SW1783 and U87 GBM cell lines were moderately sensitive to bendamustine, while A172, Mz54, T98G and U118 were resistant to bendamustine. As can be seen from FIG. 5, when bendamustine was combined with 500 nM vorinostat very similar results were achieved to those with TMZ and vorinostat, i.e. all cell lines were highly sensitive except Mz54 and T98G and while the IC₅₀ of T98G was reduced it is not the range of achievable doses in humans.

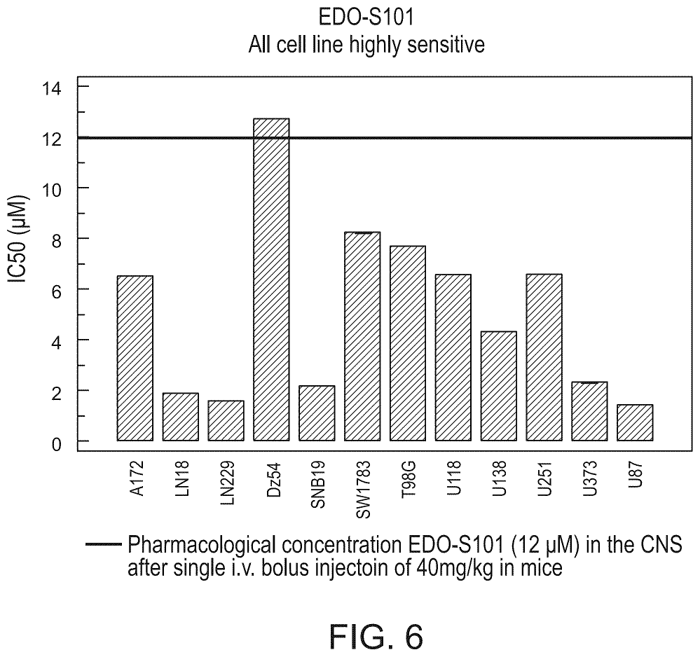

In comparison to the other single compounds and combinations, the IC₅₀ curves for the twelve tested cell lines in FIG. 6 demonstrate that all twelve cell lines including all of the MGMT positive cell lines were highly sensitive to EDO-S101. This demonstrates that EDO-S101 is a highly promising therapeutic agent against both MGMT negative and MGMT positive GBMs.

A summary of the IC₅₀ values for the different cell lines is shown in the following Table 2.

TABLE 2

| Cell | | Charac- | | EDO- | Temozol- |
|---|---|---|---|---|---|
| Line | Origin | teristics | Bendamustin | S101 | amide |
| U251MG | Sigma- | MGMT | 30.0 | 6.60 | 20.0 |
| | Aldrich | neg. | | | |
| | (09063001) | | | | |
| U87MG | ATCC | MGMT | 50.0 | 1.36 | 20.0 |
| | (HTB-14) | neg. | | | |
| T98G | ATCC | MGMT | 52.0 | 7.70 | >100 |
| | CRL-1690 | pos. | | | |
| U118MG | HTB-15 | MGMT | 35.0 | 6.61 | >100 |
| | | pos. | | | |
| U373MG | Sigma- | MGMT | 35.0 | 2.26 | 80.0 |
| (Uppsala) | Aldrich | neg. | | | |
| | (08061901) | | | | |
| Mz-54 | Goethe- | MGMT | 60.0 | 12.73 | >100 |
| | University | pos. | | | |
| | Frankfurt | | | | |
| A172 | CRL-1620 | MGMT | 55.0 | 6.45 | ~100 |
| | | neg. | | | |
| U138MG | ATCC | MGMT | 30.0 | 4.27 | >100 |
| | (HTB-16) | pos. | | | |
| LN228 | ATCC | MGMT | 35.0 | 1.55 | >100 |
| | (CRL- | neg | | | |
| | 2611) | | | | |
| SW1783 | ATCC | MGMT | 38.0 | 8.24 | 80.0 |
| | (HTB-13) | neg. | | | |
| LN18 | ATCC | MGMT | 25.0 | 1.87 | >100 |
| | (CRL- | pos. | | | |
| | 2610) | | | | |
| SNB19 | NCI | MGMT | 32.0 | 2.17 | >100 |
| | | neg. | | | |

### Example 3 In Vivo Evaluation of EDO-S101 in Murine Models for Glioblastoma Multiforme

Therapeutic activity of EDO-S101 was determined in murine brain tumour models against GBM, based on tumour growth as determined by bioluminescence imaging and survival analysis as determined by Kaplan-Meier analysis.

Murine brain tumour models were created by intracerebral injection of 3λ10⁵ luciferase-transfected GBM12 cells in athymic mice under aesthesia using a stereotactic platform. GBM12 is a MGMT negative tumour cell line. Eight-week-old athymic mice underwent minimum 7-day acclimation/quarantine prior to surgery. Surgery was performed in a laminar flow hood under sterile conditions. Tylenol 300 mg/kg PO was given for analgesia 24 hours before the surgery continuing 48 hours postoperatively. Aesthesia was achieved by inhalation of 1-2% isoflurane. After the mouse became well anesthetized, it was placed in the Kopf stereotactic instrument. A small amount of BNP antibiotic cream (a mixture of Bacitracin, Neomycin and Polymyxin) was smeared on its eyes to prevent infection and corneal damage during surgery. A strip of soft fabric was placed over the mouse's body and tail to prevent excessive heat loss during surgery. The scalp area was cleaned with a 2% solution of Betadine and dried with cotton tipped applicator. A midline sagittal incision was made in the scalp.

A small burr hole was drilled in the left skull with a surgical drill (Kopf) or a Dremel drill according to the coordinates (AP: 0.5 mm, LM: 2.5 mm) as determined by reference to the mouse brain atlas by Franklin and Paxinos. The dura mater was surgically exposed, and a 10 μl-Hamilton syringe with a 26S-gauge bevelled needle was lowered into the left cerebral hemisphere up to the depth of 3 mm and 5 μl of the 3×10⁵ luciferase-transfected GBM12 cells tumour cells was slowly infused (0.5 μl/min). The needle was left in place for 5 minutes to prevent reflux and then slowly removed. The skin was closed with wound clips. After surgery, the mice recovered in a warm environment and returned to their cages when motor activity returned. Cages were placed on top of a heating pad to minimize the loss of body heat during the recovery. The mice were monitored post-operatively at least twice a day for 5 days or until recovery is complete. EDO-S101 (60 mg/kg body weight) or bendamustine (50 mg/kg body weight) were administered via the tail vein starting at day +4 post intracerebral tumour cell implantation and then subsequently at day +11 and day +18. Limb paralysis was taken as an endpoint for survival analysis.

After intracerebral injection of the GBM cells, all the mice were subjected to bioluminescence imaging (BLI) twice a week starting at day-4 post-intracerebral injection to monitor the real-time in vivo tumour growth. BLI was conducted using a Xenogen Lumina optical imaging system (Caliper Life Sciences, Hopkinton, MA). Mice were anesthetized with isoflurane before intraperitoneal injections of luciferin at a dose of 150 mg/kg, providing a saturating substrate concentration for luciferase enzyme. Peak luminescent signals were recorded 10 minutes after luciferin injection. Regions of interest encompassing the intracranial area of signal were defined using Living Image software (Xenogen, Alameda, CA), and the total photons/s/steradian/cm2 was recorded.

ANOVA was used to determine the statistical significance of the differences between experimental groups at each time point. Kaplan-Meier survival curves were generated using Prism4 software (GraphPad Software, LaJolla CA) and the statistical difference between curves was derived with a log-rank test. P<0.05 was considered significant.

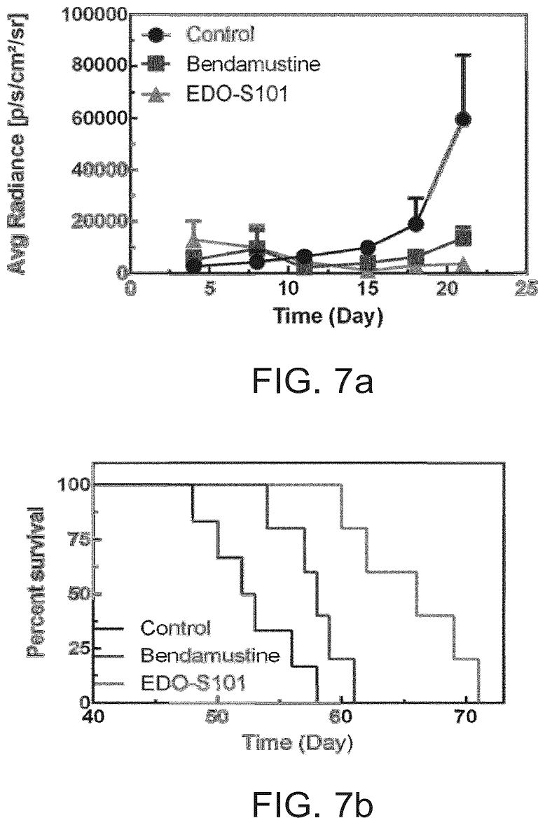

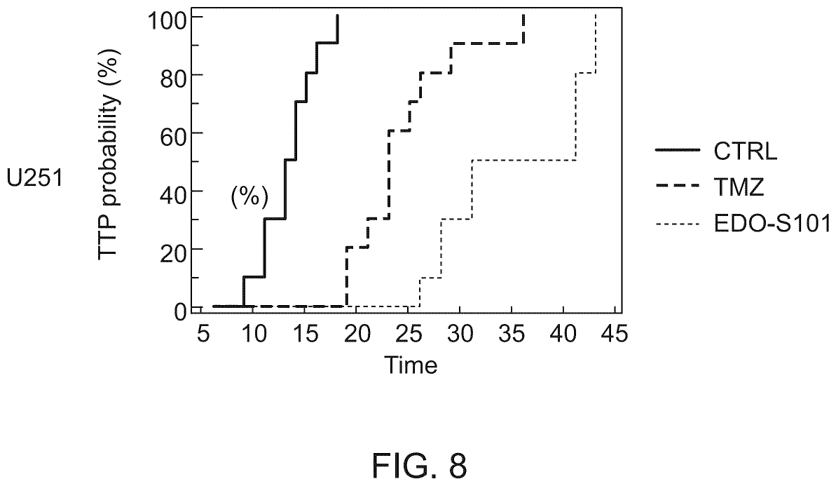

In this patient-derived xenograft model for GBM (GBM12), EDO-S101 was administered at IV 60 mg/kg weekly on day +4, +11, +18 post intracerebral implantation of tumour cells (MTD dose). Bendamustine was given at IV 50 mg/kg weekly on day +4, +11, +18 (MTD dose). EDO-S101 was found to have significant therapeutic activity with suppression of tumour growth and prolongation of survival with median survival of 66 days compared to 58 days with Bendamustine, and 52 days in no-treatment controls (see FIGS. 7a and 7b). EDO-S101 has excellent therapeutic activity against this MGMT negative glioblastoma multiforme.

The above procedure was followed in similar manner using the cell lines U87G and U251G. Once again, EDO-S101 (60 mg/kg) was administered intravenously via the tail vein, but in these experiments it was administered at days 1, 8 and 15. In place of bendamustime, TMZ was administered as a comparison at 16 mg/kg for 5 consecutive days, po. The mice were sacrificed after 28 days.

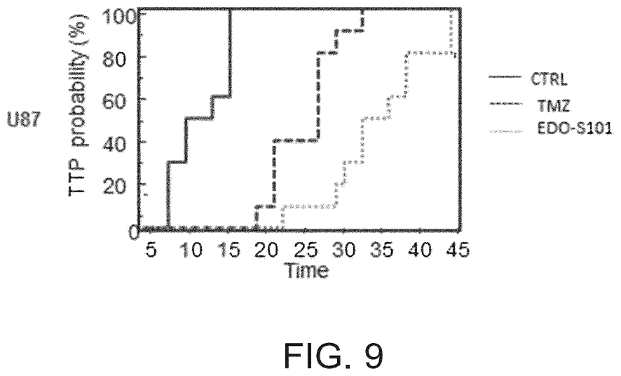

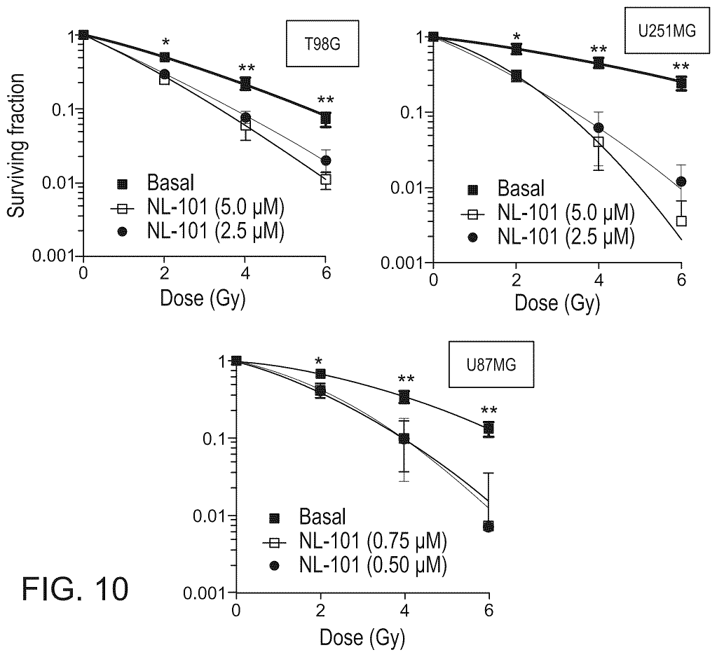

The plot of time to progression (TTP) probability (%) against time in FIG. 8 for mice having implanted U251 tumours shows that the TTP for the mice treated with EDO-S101 was significantly longer than that observed both for the control mice and those treated with TMZ. A similar significant increase in TTP was observed for mice having implanted U87 tumours, with EDO-S101 having a significantly longer TTP than both control and TMZ (see FIG. 9).

### Example 4 In Vivo Evaluation of EDO-S101 (Alone or in Combination with Radiotherapy) in Murine Models for Glioblastoma Multiforme Against Radiotherapy and Temozolamide (Alone or in Combination)

In a first experiment, U251, U87 and T98G cell lines were treated with radiotherapy alone or with radiotherapy and EDO-S101.

For clonogenic survival, exponentially growing cells (70% confluence) were cultured in regular media and treated with EDO-S101 at the appropriate concentrations, or vehicle (final DMSO concentration of 0.1%) for 24 hr. Tumour cell irradiation was done using a 6 MV linear accelerator Elekta Synergy using a clinically calibrated irradiation field of 30×30 cm. Two cm thick plates of perspex were positioned above and below the cell culture flasks completely filled with medium to compensate for the build-up effect. Non-irradiated controls were handled identically to the irradiated cells with the exception of the radiation exposure. After treatment, cells were diluted at the appropriate concentration (1,000 cells) and re-seeded into a new 100 mm tissue culture dish (in triplicate) and incubated for 14 days. At day 14 the media was removed and colonies were fixed with methanol: acetic acid (10:1, v/v), and stained with crystal violet. Colonies containing more than 50 cells were counted. The plating efficiency (PE) was calculated as the number of colonies observed/the number of cell plated. The surviving fraction was calculated as the number of colonies formed in the treated dishes compared with the number formed in the control. The survival curves were analyzed using SPSS (Chicago, IL) statistical software by means of a fit of the data by a weighted, stratified, linear regression, according to the linear-quadratic formula:

$$S ( D ) / S ( O ) = exp - ( a D + b D 2 ) .$$

For the MGMT negative U251MG glioblastoma cell line, the IC₅₀ was measured to be 6.60 μM for EDO-S101 (compared to 30 μM for bendamustin and 20 μM for temozolamide).

For the MGMT negative U87G glioblastoma cell line, the IC₅₀ was measured to be 1.36 μM for EDO-S101 (compared to 50 μM for bendamustin and 20 μM for temozolamide).

For the MGMT positive T98G glioblastoma cell line, the IC₅₀ was measured to be 7.70 μM for EDO-S101 (compared to 52 μM for bendamustin and >100 UM for temozolamide).

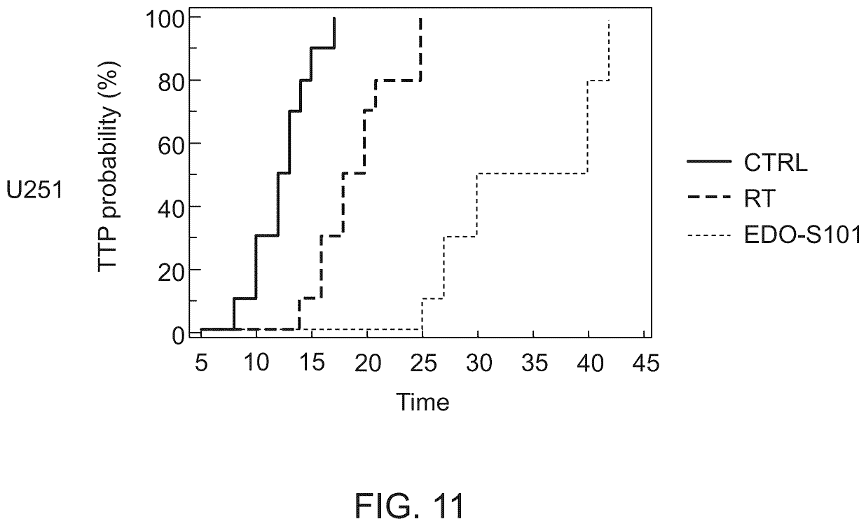

As can be seen from FIG. 10, the % survival rate for the glioblastoma cells was considerably reduced when radiotherapy was used in combination with a dose of EDO-S101 (2.5 μM or 5 μM) compared to radiotherapy alone, in all 3 GBM cell lines.

Next, adopting the procedure of Example 3, s.c. xenograft models of GBMs in mice were prepared using the GBM cell lines U251 and U87.

The U251 mice prepared as above were subjected either subjected to radiotherapy (2 Gy for 5 consecutive days), treatment with EDO-S101 (60 mg/kg intravenously at days 1, 8 and 15 of the treatment cycle) or control only. Before any irradiation mice were anesthetized with a mixture of ketamine (25 mg/ml)/xylazine (5 mg/ml). Anesthetized tumor-bearing mice received a focal irradiation at the dose of 2Gy for 5 consecutive days. Irradiation was delivered using an X-ray linear accelerator at a dose rate of 200 cGy/min at room temperature. All mice were shielded with a specially designed lead apparatus to allow irradiation to the right hind limb. Mice were kept under these conditions until all irradiation finished.

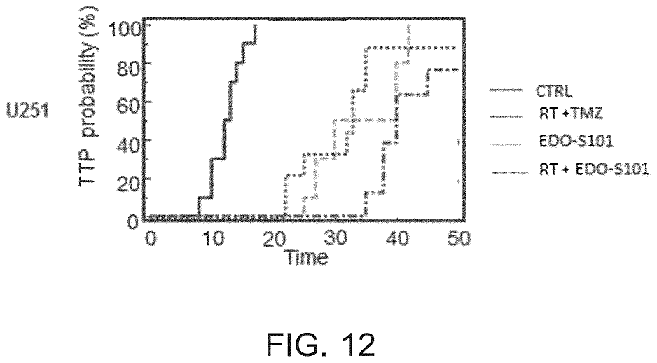

A study was made of the progression of the GBM according to the procedure of Example 3. A plot of the time to progression probability (%) against time is shown in FIG. 11. From this, it is evident that the time to progression for the mice treated with EDO-S101 is considerably longer than observed for radiotherapy-treated tumours.

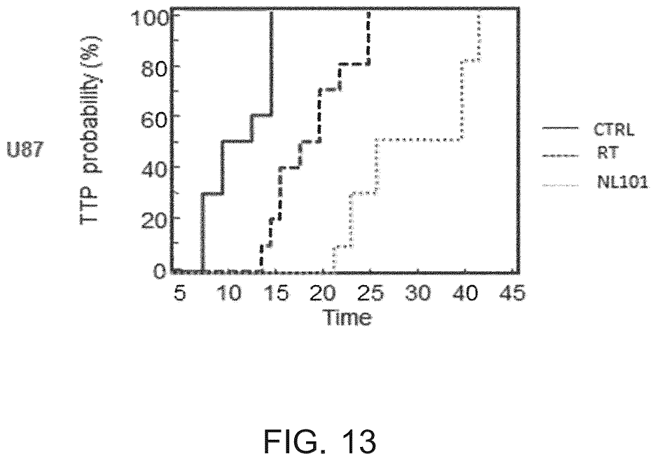

In a follow up experiment, U251 mice prepared in the same manner were either subjected to the current gold standard treatment of radiotherapy and temozolomide (2 Gy for 5 consecutive days and 16 mg/kg for 5 consecutive days, po), treatment with EDO-S101 (60 mg/kg, intravenously at days 1, 8 and 15 of the treatment cycle), treatment with EDO-S101 and radiotherapy (2 Gy for 5 consecutive days and 60 mg/kg, intravenously at days 1, 8 and 15 of the treatment cycle) or control. A plot of the time to progression probability (%) against time is shown in FIG. 12. From this, it is evident that the time to progression for the mice treated with EDO-S101 and radiotherapy is significantly longer than that observed for tumours treated with EDO-S101 alone. Furthermore, the time to progression for the combination of radiotherapy and EDO-S101 was significantly longer than that observed for tumours treated with radiotherapy and temozolomide, the current gold standard treatment.

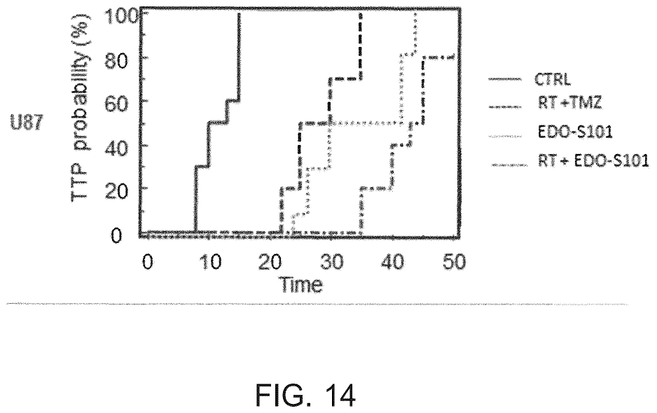

The same sequence of experiments was followed, but this time with the s.c. xenograft models of GBM in mice prepared using the GBM cell line U87. In a first experiment, the U87 mice prepared as above were subjected either subjected to radiotherapy (2 Gy for 5 consecutive days), treatment with EDO-S101 (60 mg/kg intravenously at days 1, 8 and 15 of the treatment cycle) or control only. A study was made of the progression of the GBM. A plot of the time to progression probability (%) against time is shown in FIG. 13. From this, it is evident that the time to progression for the mice treated with EDO-S101 (referred to in FIG. 13 as NL101) is considerably longer than observed for radiotherapy treated tumours.

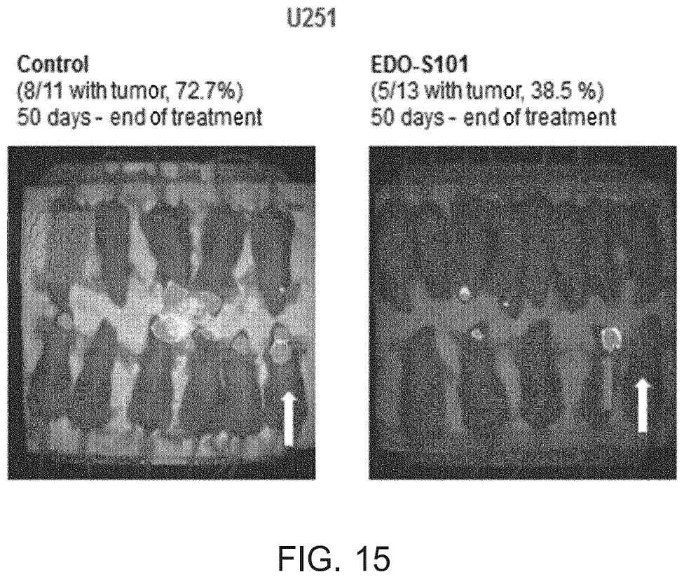

In a similar follow up experiment as that used for the U251 mice, U87 mice prepared in the same manner were either subjected to the current gold standard treatment radiotherapy and temozolomide (2 Gy for 5 consecutive days and 16 mg/kg for 5 consecutive days, po), treatment with EDO-S101 (60 mg/kg, intravenously at days 1, 8 and 15 of the treatment cycle), treatment with EDO-S101 and radiotherapy (2 Gy for 5 consecutive days and 60 mg/kg, intravenously at days 1, 8 and 15 of the treatment cycle) or control. A plot of the time to progression probability (%) against time is shown in FIG. 14. From this, it is evident that the time to progression for the mice treated with EDO-S101 and radiotherapy is significantly longer than that observed for tumours treated with EDO-S101 alone. Furthermore, the time to progression for the combination of radiotherapy and EDO-S101 was significantly longer that observed for radiotherapy and temozolomide, the current gold standard treatment. It should also be noted that the time to progression observed for the U87 mice treated with EDO-S101 alone was actually greater than that achieved with the combined radiotherapy and temozolomide treatment.

The time to progression of the tumours was increased from approximately 17-18 days for the control for the U251G mouse xenograft model, to 42 days with a combination of radiotherapy and temozolamide to over 50 days for EDO-S101 alone (significance P=0.924) to significantly over 50 days for a combination of EDO-S101 and radiotherapy (significance P=0.0359).

It was found that the time to progression of the tumours was increased from approximately 15 days for the control for the U87G mouse xenograft model, to 35 days with a combination of radiotherapy and temozolamide to 40 days for EDO-S101 alone (significance P=2372) to significantly over 50 days for a combination of EDO-S101 and radiotherapy (significance P=0.0001).

### Example 5 Histological Evaluation of Tumours: Orthotopic Model of U251-Luciferase Transfected Cells

Mice isotopically transfected with U251-luciferase in accordance with the procedure of Example 3 were treated with radiotherapy (2 Gy for 5 consecutive days), temozolomide (16 mg/kg for 5 consecutive days, po), radiotherapy and temozolomide (2 Gy for 5 consecutive days and 16 mg/kg for 5 consecutive days, po), EDO-S101 (60 mg/kg, intravenously at days 1, 8 and 15 of the treatment cycle) or control vehicle.

Intracranial tumour growth was monitored with the Hamamatsu imaging system (Caliper Life Sciences, Hopkinton, MA, USA). Mice were anesthetized with 2% to 4% isofluorane (Baxter, Deerfield, IL, USA) followed by intraperitoneal injections of 150 mg/kg d-luciferin (In Vivo Imaging Solutions). Five animals were measured at the same time and the luminescent camera was set to 1 minute exposure, medium binning, 1 f/stop, blocked excitation filter, and open emission filter. The photographic camera was set to 2 s exposure, medium binning, and 8 f/stop. The field of view was set to 22 cm to capture five mice at once. Serial images were taken on a weekly basis using identical settings. Bioluminescence intensity was quantified using the Living Image software (Caliper Life Sciences).

Before any irradiation mice were anesthetized with a mixture of ketamine (25 mg/ml)/xylazine (5 mg/ml). Anesthetized tumour-bearing mice received a focal irradiation at the dose of 2Gy for 5 consecutive days. Irradiation was delivered using an X-ray linear accelerator at a dose rate of 200 cGy/min at room temperature. All mice were shielded with a specially designed lead apparatus to allow irradiation to the right hind limb. Mice were kept under these conditions until all irradiation finished.

All images were obtained in the transverse plane using the following sequences: transverse T2-weighted turbo spin-echo (TSE) sequence (repetition time [TR] msec/echo time [TE] msec) 6766/120, number of signal acquired 4, matrix of 192×192) applied with a section thickness of 0.9 mm, an intersection gap of 0.0 mm, and a flip angle of 160°. The field of view was 36×60 mm², which included the tumour in its entirety with a resultant voxel size of 0.3×0.3×1.0 mm³.

Continuous variables were summarized as mean and standard deviation (SD) or as median and 95% CI for the median. For continuous variables not normally distributed, statistical comparisons between control and treated groups were established by carrying out the Kruskal-Wallis Tests. For continuous variables normally distributed, statistical comparisons between control and treated groups were established by carrying out the ANOVA test or by Student t test for unpaired data (for two comparisons).

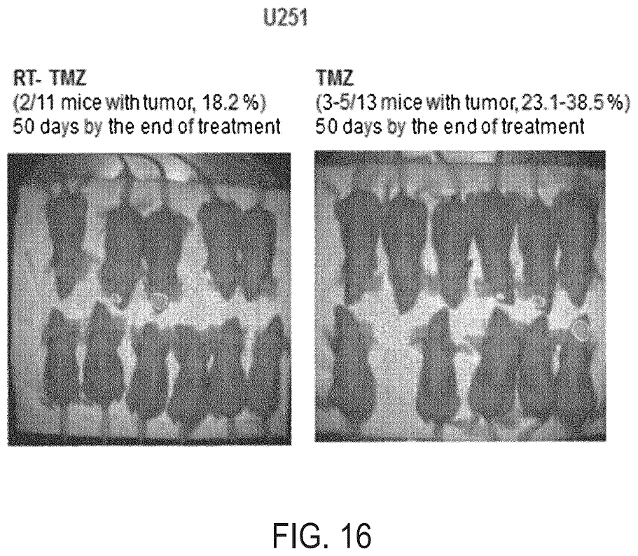

50 days after beginning of the different treatment regimes, the mice were sacrificed and the final intracranial lesions were visualized in the mice subjected to treatment with the control, EDO-S101, temozolomide, and radiotherapy and temozolomide. The results are shown in FIGS. 15 and 16. Similar results were achieved with both the EDO-S101 and temozolomide studies, both showing 5 out of 13 mice with tumours of some grade (38.5%) compared to 8 out of 11 (72.7%) in the control. However, only 1 of the 13 of the EDO-S101 treated mice displayed a large lesion, while 2 of the 13 temozolomide treated mice displayed large lesions. In the radiotherapy and temozolomide study, only 2 of the 11 mice (18.2%) displayed lesions at the end of the study, although both of these were large lesions. It can be concluded from this that EDO-S101 is highly effective in preventing spread of GBMs.

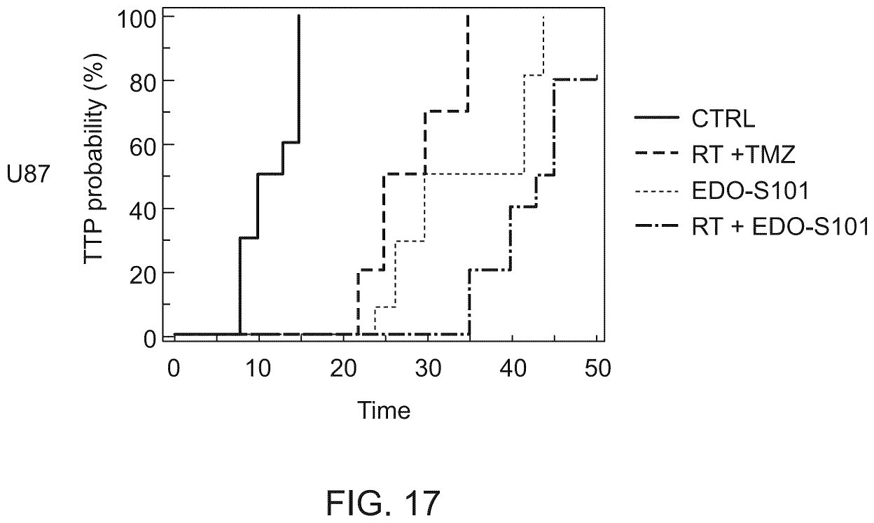

The effectiveness of EDO-S101 in preventing spread of GBMs is further emphasized in FIG. 17, showing a plot of survival probability (%) against time (days). The survival probability for the mice treated with EDO-S101 was significantly greater than that for those treated with either radiotherapy or temozolomide. Only the mice treated with a combination of radiotherapy and temozolomide showed a higher overall survival probability than EDO-S101 alone.

### Example 6 In Vivo Evaluation of EDO-S101 in Murine Models for Primary CNS Lymphoma

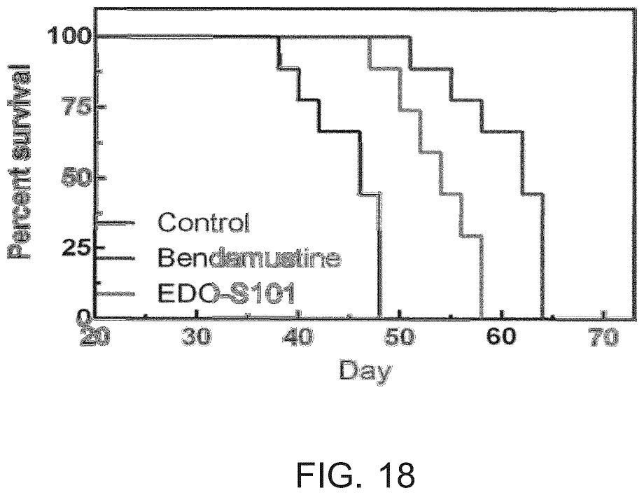

The procedure of Example 3 was repeated, except the murine models were created with 1×10⁵ luciferase-transfected OCI-LY10B lymphoma cells to create a model of primary CNS lymphoma. EDO-S101 (60 mg/kg body weight), bendamustine (50 mg/kg body weight) and control was administered intravenously via a tail vein to separate groups of the test mice on days +4, +11 and +18 post intracerebral implantation of the OCI-LY10B lymphoma cells. Both EDO-S101 and bendamustine significantly suppressed tumour growth and prolonged the survival with median survival of 62 days and 54 days respectively compared to 48 days in no-treatment controls (see FIGS. 18a and 18b). EDO-S101 therefore appears to be a promising treatment for primary CNS lymphoma.

### Example 7 In Vivo Evaluation of EDO-S101 in Murine Models for Triple Metastatic Breast Cancer of the Brain

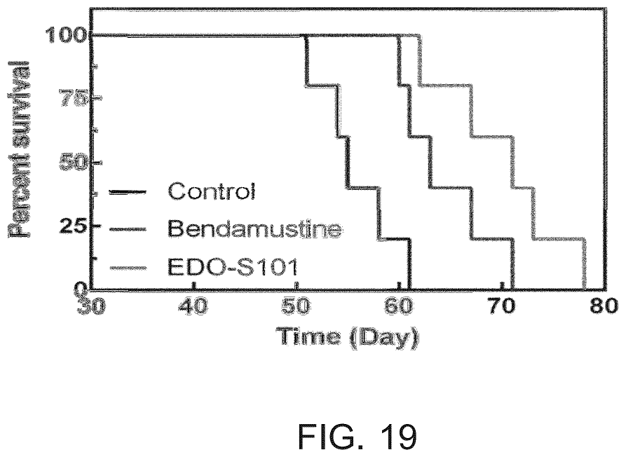

The procedure of Example 3 was repeated, except the murine models were created with 1×10⁵ luciferase-transfected MB-468 breast cancer cells to create a model of primary CNS lymphoma. EDO-S101 (60 mg/kg body weight), bendamustine (50 mg/kg body weight) and control was administered intravenously via a tail vein to separate groups of the test mice in a single dose on day+4 post intracerebral implantation of the MB-468 breast cancer cells. EDO-S101 showed significant therapeutic activity with suppression of tumour growth and prolongation of survival with median survival of 71 days compared to 62 days for bendamustine and 55 days for no-treatment controls (see FIGS. 19a and 19b). EDO-S101 therefore appears to be a particularly promising treatment for metastatic brain cancer.

In conclusion, the experiments demonstrate that the ability of EDO-S101 to pass through the blood-brain barrier is very good. This makes it a promising candidate for treatment of brain cancers. The experimental data further shows that it is active not only against MGMT negative GBMs but also MGMT positive GBMs, making it highly promising as a therapeutic for treatment of MGMT positive GBMs and other MGMT positive astrocytic brain tumours as no therapy has yet been developed for these. It also shows that it significantly prolongs median survival in cases of both primary CNS lymphoma and metastatic brain cancers, again making it a very promising therapeutic candidate for both conditions. The data also show that when EDO-S101 is administered in combination with radiotherapy then it shows significantly improved activity compared to EDO-S101 alone in the treatment of GBM.

1.-54. (canceled)

55. A method of treating an O⁶-methylguanine-DNA methyltransferase (MGMT) negative Glioblastoma Multiforme (GBM) in a patient in need thereof, the method comprising administering to said patient a compound of formula I or a pharmacologically acceptable salt thereof:

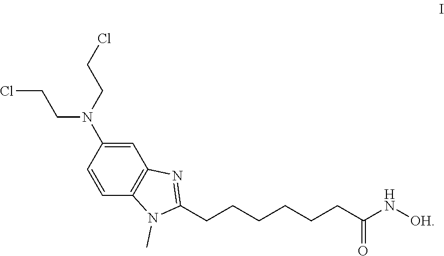 [SMILES: Cn1c(CCCCCCC(=O)NO)nc2cc(N(CCCl)CCCl)ccc21]

56. The method according to claim 55, wherein the pharmacologically acceptable salt of the compound of formula I is a hydrochloride, hydrobromide, hydroiodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate or acetate salt.

57. The method according to claim 55, wherein the compound of formula I or pharmacologically acceptable salt thereof is administered intravenously to the patient in need thereof.

58. The method according to claim 55, wherein the compound of formula I or pharmacologically acceptable salt thereof is administered intravenously to the patient in need thereof at a dosage level of from 0.1 mg/kg to 70 mg/kg body weight of the patient.

59. The method according to claim 55, wherein the compound of formula I or pharmacologically acceptable salt thereof is administered intravenously to the patient in need thereof at a dosage level of from 0.5 mg/kg to 50 mg/kg body weight of the patient.

60. The method according to claim 55, wherein the compound of formula I or pharmacologically acceptable salt thereof is administered intravenously to the patient in need thereof on at least one day selected from Day 1, Day 8, and Day 15 of a treatment cycle.

61. The method according to claim 55, wherein the compound of formula I or pharmacologically acceptable salt thereof is administered intravenously to the patient in need thereof on Day 1 and/or Day 15 of a treatment cycle.

62. The method according to claim 55, wherein the compound of formula I or pharmacologically acceptable salt thereof is administered intravenously to the patient in need thereof on Day 1 only of a treatment cycle.

63. The method according to claim 55, wherein the compound of formula I or pharmacologically acceptable salt thereof is contained in a medicament, wherein said medicament further comprises a liquid carrier selected from the group of consisting of polyethylene glycol, cyclodextrin and a fatty oil.

64. The method according to claim 55, wherein the compound of formula I or pharmacologically acceptable salt thereof is contained in a medicament further comprising cyclodextrin.

65. The method according to claim 55, wherein the compound of formula I or a pharmacologically acceptable salt thereof is administered in combination with radiotherapy.

66. The method according to claim 65, wherein said radiotherapy is given to the patient in need thereof at a dose of 1 to 5 Gy over 5 consecutive days.

67. The method according to claim 64, wherein the patient in need thereof is receiving concurrently said radiotherapy with treatment with the compound of formula I or pharmacologically acceptable salt thereof.

68. The method according to claim 64, wherein the patient in need thereof is given said radiotherapy prior to treatment with the compound of formula I or pharmacologically acceptable salt thereof.

69. The method according to claim 55, wherein the patient has received a surgery treatment followed by a treatment of temozolomide.
